(19) United States
(12) Patent Application Publication
(10) Pub. No.: US 2022/0296791 A1
(43) Pub. Date: Sep. 22, 2022

(54) DISPOSABLE SURGICAL DRAIN BULB FOR THE SECURE COLLECTION OF EXUDATE

(71) Applicant: Proximate Concepts LLC, Fort Lee, NJ (US)

(72) Inventor: Paul Rosenberg, Saddle River, NJ (US)

(21) Appl. No.: 17/205,290

(22) Filed: Mar. 18, 2021

(51) Int. Cl. A61M 1/00

(52) U.S. Cl. CPC A61M 1/0019; A61M 1/008; A61M 1/0094; A61M 2205/273

(57) ABSTRACT

Embodiments of the present invention describe a disposable drain bulb for the collection of fluid and other exudate. A disposable drain bulb in accordance with one embodiment comprises a drain interface with a one way valve for connection to a drain line through which the drain interface receives fluid and other exudate, the drain interface sealed with a capsule, and a drain body forming an internal reservoir into which the drain interface passes and that contains an absorbable material under vacuum while the drain interface remains in a closed state.

### COPYRIGHT NOTICE

A portion of the disclosure of this patent document contains material, which is subject to copyright protection. The copyright owner has no objection to the facsimile reproduction by anyone of the patent document or the patent disclosure, as it appears in the Patent and Trademark Office patent files or records, but otherwise reserves all copyright rights whatsoever.

### FIELD OF THE INVENTION

The inventions described herein generally relate to drain bulbs for use with surgical drains. More specifically, inventions disclosed and described herein relate to disposable surgical drain bulbs that use an absorbable structure disposed within the interior of the bulb to assist with the collection of fluid and other exudate entering the drain bulb from a drain connected thereto.

### BACKGROUND OF THE INVENTION

Surgical drains are temporary implants that allow for the removal of a wide variety of fluids from a wound or body cavity. Surgical drains can help the healing process by removing blood, blood products such as serum, retained irrigation fluids, inflammatory mediators, bacteria, foreign materials, and necrotic tissue. Drains are also useful in relieving pressure that can impair perfusion or cause pain, thereby decreasing morbidity and reducing inflammation, as well as enable the easy sampling of fluid during healing and addressing complications associated with dead space.

Drains are classified by characteristics of the various systems: open or closed and passive or active. Typically, passive drains are open systems and made of latex, polypropylene, rubber, etc. They primarily work by way of capillary action, gravity, overflow, or fluctuations of pressure gradients caused by body movement. Active drains, by contrast, are closed systems because they rely on negative pressure that is created by the drain or a dedicated negative pressure source. Negative pressure increases the efficiency of active over passive drains, enables placement of the drain exit in any position, allows for the removal of fluid against gravity if necessary, and can be used to collapse dead space.

FIG. 1A illustrates one embodiment of a Jackson-Pratt® surgical drain (“Jackson-Pratt drain” or JP drain”) in accordance with the prior art that is an exemplary closed, active surgical drain system well known to those of ordinary skill in the art. The Jackson-Pratt drain and bulb system is a type of closed suction device comprising a small, translucent plastic or rubber bulb 100 operative to provide suction, which is attached to a length of drain tube (not pictured). The drain tube comprises a thin, flexible rubber tube, the open, free end of which is inserted within the surgical wound before the surgical field is closed. A small incision, or cut, is made in the skin for the tube to enter and in some instances the skin and tube are sutured together to ensure the drain does not move from under the skin; the drain bulb 100 remains outside the patient's body. Petroleum jelly or similar ointment may be applied to the exit site to seal the wound. Various types of drain tubes are known for use in moving fluid and exudate collecting in a wound into a reservoir portion 102 of the JP drain 100.

The drain tube is connected to the bulb portion 100 of the JP drain by way of a one way collection port 104. Being a one way valve, fluid and exudate may flow through the collection port 104 and into the interior reservoir 102 of the bulb 100, also referred to as a “grenade”, by way of an optional input line 110. In other instances, the collection port 104 allows for the fluid and exudate to flow from the drain directly into the reservoir 102. Also provided is a discharge port 106, which is a two way valve that allows for the discharge of fluid and exudate that has collected in the reservoir 102 over time. As is explained herein, to maintain suction that the bulb 100 provides, a stopper 108 is provided to seal the discharge port.

Turning to FIG. 1B, air is evacuated from the drain bulb by first opening the discharge port 106, gently squeezing the bulb 100 to expel the air inside and closing the stopper 108 to seal the discharge port 106. When air is removed from the bulb 100, the bulb will physically flatten and there will be a suctioning effect. This suction or negative pressure draws fluids, gas, and other exudate out from inside the body of the patient and into the interior volume 102 of the bulb 100. When utilizing a Jackson-Pratt drain it is important to sufficiently squeeze the air out of the bulb so that the suction, or negative pressure, continues to pull fluid from the body, failure to properly reset the vacuum of the bulb 100 leads to diminished operation and efficacy. As a closed system, all fluids and other exudate exiting the body by way of the drain tube are collected within the bulb portion 102 of the JP drain without the need for an outside source of suction or negative pressure, e.g., an external mechanical source of negative pressure.

With the collection port 104 affixed to the flexible tubing of the drain, the bulb 100 expands slowly over time and, as it does, creates a vacuum in the drain tube. By means of this vacuum, exudate present in the surgical wound is evacuated into the interior 102 of the bulb 100. When the interior 102 is full, or on a regular schedule, a nurse or technician opens the discharge port, empties the bulb 100 through the discharge port 106, recompresses the bulb 100, closes the discharge port 106 by affixing the stopper 108, and the cycle repeats. When emptying fluid and exudate that has collected inside 102 the bulb 100, the nurse or technician ordinarily observes the type and character of the fluid collected. For example, taking note of the color, odor, consistency, and volume, noting this data in the patient's chart.

Depending on the type of surgical procedure, multiple of drains may be installed in one surgical wound and the drains may be installed for periods of time ranging from a few days to weeks (or months). As such, there are many situations where patients are discharged from a hospital or rehabilitation facility with one more surgical drains intact, requiring the patient to care, empty, and otherwise engage in daily care of the drain. A serious problem that patients frequently encounter involves emptying the filled bulb portion of the JP drain and properly resetting the vacuum prior to reconnection to the drain line.

As discussed above, the patient must engage in the procedure carried out by trained hospital personnel, which includes opening the stopper 108 on the discharge port 106 and emptying the exudate into a discharge receptacle, which may expose the untrained patient or others to bacteria or viruses contained in the exudate. Similarly, the untrained patient may be called upon to reset the suction of the bulb 100. If done incorrectly, this may cause unintended discharge of exudate outside the bulb 100 or decrease the generally efficacy with which the bulb removes exudate from the body of the patient, which may have further deleterious health consequences for the patient (or others exposed to the exudate) and may be associated with suboptimal outcomes for surgical procedures.

Accordingly, there is a need for new and improved surgical drainage system that provides for the benefits associated with closed, active drainage systems, but without the drawbacks associated with such drains as presently known in the art.

### SUMMARY OF THE INVENTION

Embodiments of the present invention are directed towards a disposable drain bulb for the collection of fluid and other exudate that provides the benefits associated with closed, active drainage systems, but without the drawbacks associated with such drains as presently know in the art. A disposable drain bulb in accordance with one embodiment of the present invention comprises a drain interface with a one way valve for connection to a drain line through which the drain interface receives fluid and other exudate, the drain interface sealed with a capsule.

The disposable drain bulb in accordance with the present embodiment further comprises a drain body, the drain body forming an internal reservoir into which the drain interface passes and that contains an absorbable material under vacuum while the drain interface remains in a closed state. According to one embodiment, the absorbable material comprises a sponge. The absorbable material in accordance with one embodiment provides structure that supports the collection of fluid in the internal reservoir, which is surrounded by the polymer wall(s) of the drain body, the purpose of which in combination is to maintain the vacuum and contain any fluid beyond what is captured by the absorbable material. Moreover, in its compressed form (deformed structure) under vacuum conditions, the absorbable material is not functional, as the air pockets are unused and unavailable to collect any exudate. As the structure regains its native shape (e.g., fully expanded, either by air or the collection of fluid), the absorbable material continues to provide an absorbable function until the full native structure is reached, at which time it ceases to collect fluid.

A capsule may seal the drain interface and may have one or more micro-perforations such that the one or more micro-perforations rupture in the presence of sufficient pressure. In response to the presence of sufficient pressure at the capsule causing the one or more micro-perforations to rupture, the drain interface enters an open state, allowing fluid and exudate to flow through the drain interface and into the internal reservoir. The capsule may further contain a fluid therein such that the fluid is pulled in the drain body when the one or more micro-perforations rupture. The presence of the fluid in the drain bodying provides an indication that the drain is open and should be connected to a drain line.

In accordance with an alternative embodiment, a disposable drain bulb for the collection of fluid and other exudate comprises a drain interface with a one way valve for connection to a drain line through which the drain interface receives fluid and other exudate, the drain interface sealed with an actuatable valve. The disposable drain bulb in accordance with the present embodiment further comprises a drain body, the drain body forming an internal reservoir into which the drain interface passes and that contains an absorbable material, which may comprise a sponge, under vacuum while the drain interface remains in a closed state. The actuatable valve moves to an open state in the presence of sufficient torque to open the valve.

### BRIEF DESCRIPTION OF THE DRAWINGS

The invention is illustrated in the figures of the accompanying drawing which are meant to be exemplary and not limiting, in which like references are intended to refer to like or corresponding parts, and in which:

FIG. 1A presents a line drawing that illustrates a Jackson-Pratt® surgical drain in a first state according to the prior art;

FIG. 1B presents a line drawing that illustrates a Jackson-Pratt® surgical drain in a second state according to the prior art;

FIG. 2 presents a line drawing that illustrates a top down view of an absorption drain according to one embodiment of the present invention;

FIG. 3 presents a line drawing that illustrates an edge view of an absorption drain according to one embodiment of the present invention;

FIG. 4 presents a line drawing that illustrates a top down view of an absorption drain according to another embodiment of the present invention; and

FIG. 5 presents a line drawing that illustrates a detailed view of a drain interface of an absorption drain according to one embodiment of the present invention.

### DETAILED DESCRIPTION OF EMBODIMENTS OF THE INVENTION

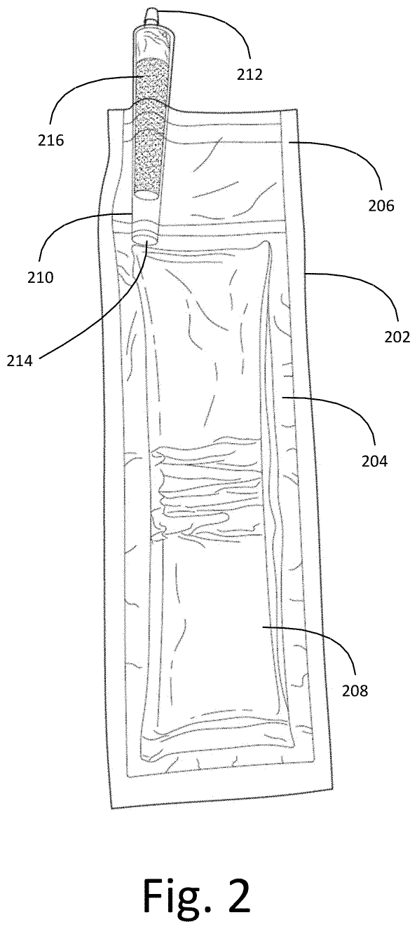

In order to avoid drawbacks associated with closed, active surgical drains, embodiments of the invention provide for a disposable drain system that utilizes an absorbable material as an insert in the drain to absorb fluid and other exudate as the vacuum created by attaching the drain to a drain line pulls such materials into the drain body or interior of the drain. FIG. 2 presents a top down view of one embodiment of such an improved absorbable surgical drain. In accordance with the embodiment presented in FIG. 2, an improved, disposable absorbable surgical drain comprises a drain body 202 and a drain interface 210. While the drain interface 210 remains sealed, the interior reservoir 204 within the drain body 202 is vacuum sealed. All air is removed from the interior reservoir 204 such that opening the drain interface 210 creates a vacuum on a drain line connected thereto (not pictured), which has the effect of drawing fluid and other exudate from the body of the patient to the interior reservoir 204 by way of the drain line.

According to the present embodiment, the drain body 202 comprises a plurality of polymer sheets that are welded 206 together to create the interior reservoir 204 for holding fluid and other exudate exiting the body. Placed within the interior reservoir 204 is a portion of absorbable material 208. The absorbable material 208 may comprise any material or combination of materials known to those of skill in the art to hold or otherwise contain a volume of fluid entering the interior reservoir 204. In accordance with one embodiment, the absorbable material 208 comprises a sponge that remains under vacuum while the drain interface 210 is in its closed position. When the drain interface 210 is open, air pockets within the absorbable material 208 may expand, filling with gasses, fluids, and other exudate that are entering the interior reservoir 204, having the effect of increasing the overall capacity of fluid volume that that body 202 is otherwise capable of holding therein. It should be noted that the drain body 202 may comprise additional geometries and constructions, e.g., formed as a sphere or teardrop from a single sheet of polymer.

The drain interface 210 passes through any welds 206 or seams in the edge or perimeter of the drain body 202 so as to provide a pathway out of the drain interface 214 and into the internal reservoir, with such seams or edges welded or otherwise sealed against the drain interface 210 to maintain the integrity of the body. The drain interface 210 comprises a one way valve 212 that is operative to communicate with a drain line, through which fluid and other exudate may exit the body of the patient, flow through and exit 214 the drain interface 210 into the interior reservoir 204 of the drain body 202.

According to the present embodiment, the vacuum is maintained within the interior reservoir 204 by sealing the drain interface with a capsule 216 or similar plug that may be ruptured upon the application of sufficient pressure. In such embodiments, the walls of the drain interface 210 are of sufficient flexibility to allow communication of pressure from an outside surface of the drain interface 210 to the interior thereof. In the presence of sufficient pressure to the outside surface of the drain interface 210, the capsule 216 ruptures, which may result in any fluid contained therein whisking into the interior reservoir 204, which is under vacuum, for absorption by the absorbable material 208. Whisking of the fluid contained in the capsule in the interior reservoir may serve as an indicator that the disposable drain bulb is active (open) and ready for connection to a drain line. Colored fluid may be used to achieve the desired effect.

The capsule 216 may be constructed according to one embodiment of a polymer and is sufficiently sized to seal the drain interface 210 and maintain the vacuum in the interior reservoir 204. Having micro-perforations or other imperfections along various sides thereof, pressure communicated to the capsule 216 causes the micro-perforations to rupture, breaking the seal so as to open the drain interface 210 and allow fluid to enter the interior reservoir 204 for storage. As the absorbable material expands 208, vacuum at the one way valve 212 continues to pull in fluid and exudate for storage in the absorbable material 208 and reservoir body 204 generally until the reservoir body 204 reaches its maximum volume. At this point there is insufficient vacuum to pull additional fluid down the drain line and, since the one way valve 212 does not discharge fluid back into the drain line, the drain 202 must be replaced.

By contrast with prior art drains, such as the Jackson-Pratt drain described above, embodiments of the disposable drain bulbs described herein are fully disposable once full, eliminating the need to discharge the contents of the drain, reset the vacuum, and reattach the drain body to the drain line. Instead, the nurse or patient simply disconnects the one way valve 212 from the drain line and attaches a new drain body 202. Once the new one way valve 212 is attached, the capsule 216 is ruptured or the seal is otherwise interrupted by release of a valve, allowing the vacuum to begin pulling exudate into the interior reservoir 204 for storage by the absorbable material 208 and interior body 204 generally.

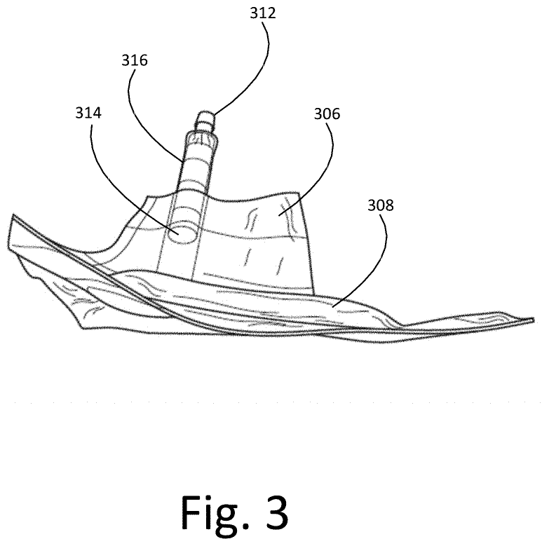

FIG. 3 presents an edge view of the embodiment of an absorption drain as introduced by FIG. 2. In accordance with the view of FIG. 3, the drain interface 316 is sealed, thereby maintaining the interior reservoir of the drain body 306 in a vacuum state. As such, the body and absorbable material 308 contained therein are generally flat as all air has been removed from the drain body 306 prior to sealing the drain interface 316, which, as set forth in FIG. 2, may be through the use of breakable capsule 314 that seals the drain/bulb interface. The drain interface 316 passes through an edge, seam, or other opening in the body 306, with the drain body 306 sealed or otherwise welded to the drain interface 316 to maintain the integrity of the drain body 306, e.g., maintain the absorbable material 308 under vacuum. Once a drain line is connected to the one way valve 312 and the drain interface 316 opened, for example, by rupturing the capsule 314, the disposable drain bulb creates vacuum on the drain line to pull fluid through the drain interface 316 and into the interior reservoir of the drain body 306 for absorption by the absorbable material 308.

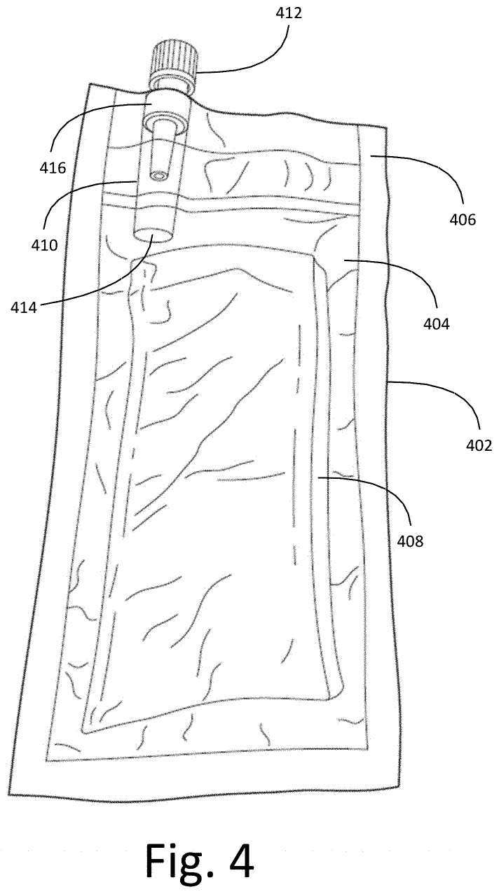

FIG. 4 presents an alternative embodiment of the absorption drain presented in FIGS. 2 and 3, which also comprises a drain body 402 and a drain interface 410. While the drain interface 410 remains sealed, the interior reservoir 404 within the drain body 402 is vacuum sealed. All air is removed from the interior reservoir 404, which may be accomplished during manufacture, such that opening the drain interface 410 creates a vacuum on a drain line connected thereto (not pictured). This has the effect of drawing fluid and other exudate from the body to the interior reservoir 404 by way of the drain line.

According to the present embodiment, the drain body 402 comprises a plurality of polymer sheets that are welded 406 together to create the interior reservoir 404 for holding fluid and other exudate exiting the body. Placed within the interior reservoir 404 is a portion of absorbable material 408. The absorbable material 408 may comprise any material or combination of materials known to those of skill in the art to hold or otherwise contain a volume of fluid entering the interior reservoir 404. In accordance with one embodiment, the absorbable material 408 comprises a sponge that remains under vacuum while the drain interface 410 is in its closed position. When the drain interface 410 is open, air pockets within the absorbable material 408 may expand, filling with gasses, fluids, and other exudate that are entering the interior reservoir 404, having the effect of increasing the overall capacity of fluid volume that that drain body 402 is otherwise capable of holding therein. It should be noted that the drain body 402 may comprise additional geometries and constructions, e.g., formed as a sphere or teardrop from a single sheet of polymer.

The drain interface 410 passes through any welds 406 or seams in the edge of the drain body 402 so as to provide a pathway out of the drain interface 414 and into the internal reservoir 404, with such seams or edges welded or otherwise sealed against the drain interface 410 to maintain the integrity of the drain body 402. The drain interface 410 comprises a one way valve 416 that is operative to communicate with a drain line, through which fluid and other exudate may exit the body of the patient, flow through the exit of the drain interface 414, and into the interior reservoir 404 of the drain body 402. The vacuum is maintained within the interior reservoir 404 by sealing the drain interface 410 (the point at which the drain interfaces the bulb) with an actuatable value 412 valve set to a closed position, which may move to an open state in the presence of sufficient torque to open the valve.

In accordance with one embodiment, the actuatable value 412 works in conjunction with the one way valve 416 to allow fluid or exudate to only flow into the internal reservoir 404 of the drain body 402 when the valve 412 is in an open position. Applying sufficient torque rotates the actuatable valve 412 into an open position or state so as to open the drain interface 410 and allow fluid to enter the interior reservoir 404 of the drain body 402 for storage. As the absorbable material 408 expands, vacuum at the one way valve 416 by way of the actuatable valve 412 continues to pull in fluid and exudate by way of the drain line for storage in the absorbable material 408 and reservoir body 404 generally until the reservoir body 404 reaches its maximum volume. At this point there is insufficient vacuum to pull additional fluid down the drain line and, since the one way valve 416 does not discharge fluid back into the drain line, the drain body 402 must be replaced.

Like the embodiment of FIG. 2, the present embodiment is fully disposable once reaching its maximum volume, thereby eliminating the need to discharge the contents of the drain, reset the vacuum, and reattach the drain body to the drain line. Instead, the nurse or patient simply disconnects the actuatable valve 412 from the drain line and attaches a new drain body 402. Once the new actuatable valve 412 is attached, sufficient torque is applied to open the actuatable valve 412. Once open, the vacuum begins pulling exudate into the interior reservoir 404 for storage by the absorbable material 408 and interior body 404 generally.

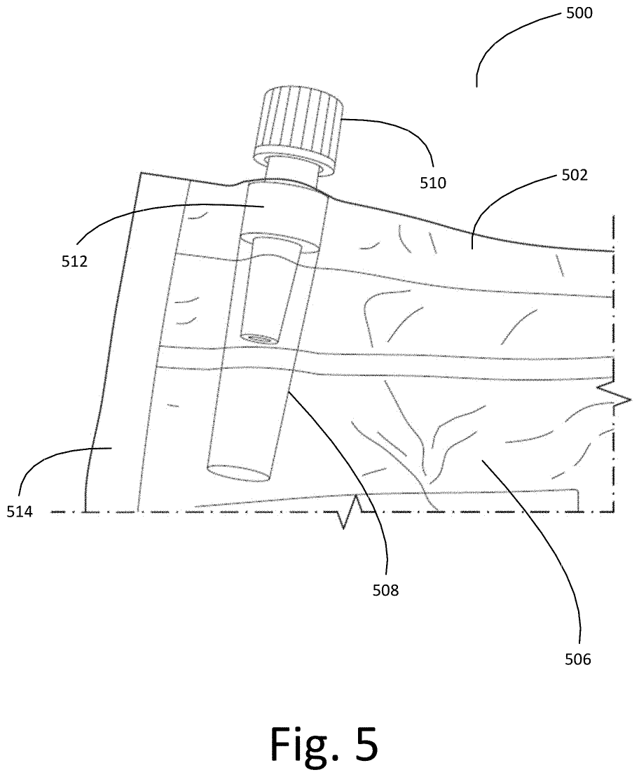

FIG. 5 presents a detailed view of the drain interface described in FIG. 4 for interfacing a disposable drain bulb in accordance with embodiments of the present invention with a drain line. As can be seen from the present embodiment, the drain interface 508 interfaces with the disposable drain bulb formed by two sheets of polymer welded 502 together, thereby creating or otherwise defining an interior reservoir 506. The drain interface 508 passes through an opening created in one of the welds 502 to create an ingress path into the interior reservoir 506 for fluid and other exudate entering the drain interface 508 by way of a drain line (not pictured). A given weld 502 through which the drain interface passes is similarly welded or sealed against the drain interface 508 to maintain the integrity of the disposable drain bulb 500.

As can be seen from the embodiment of FIG. 5, the drain interface 508 comprises a one way valve 512 that is in communication with the interior walls of the drain interface 508 so as to form a seal. An actuatable valve 510 controls flow of exudate into the one way valve 512 and interior reservoir 506 of the adsorbable drain 500. In the presence of sufficient torque, the actuatable valve 510 twists to transition from a closed state to an open state, breaking the seal and applying a vacuum created by the interior 506 of the disposable drain bulb 500 on the drain line. As the one way valve 512 does not allow exudate to flow out of the interior reservoir 506, there is no need to close the actuatable valve 510 when removing the disposable drain bulb 500 from a drain line for disposal.

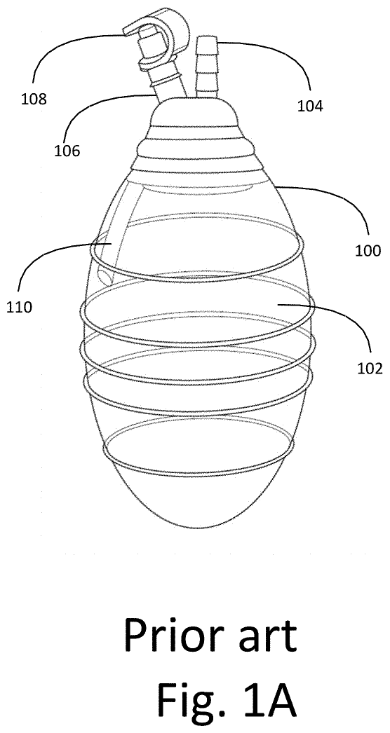

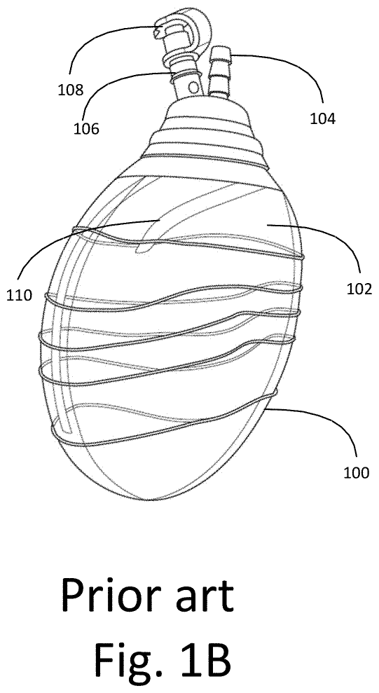

FIGS. 1 through 6 are conceptual illustrations allowing for an explanation of the present invention. Notably, the figures and examples above are not meant to limit the scope of the present invention to a single embodiment, as other embodiments are possible by way of interchange of some or all of the described or illustrated elements. Moreover, where certain elements of the present invention can be partially or fully implemented using known components, only those portions of such known components that are necessary for an understanding of the present invention are described, and detailed descriptions of other portions of such known components are omitted so as not to obscure the invention. In the present specification, an embodiment showing a singular component should not necessarily be limited to other embodiments including a plurality of the same component, and vice-versa, unless explicitly stated otherwise herein. Moreover, applicants do not intend for any term in the specification or claims to be ascribed an uncommon or special meaning unless explicitly set forth as such. Further, the present invention encompasses present and future known equivalents to the known components referred to herein by way of illustration.

The foregoing description of the specific embodiments will so fully reveal the general nature of the invention that others can, by applying knowledge within the skill of the relevant art(s) (including the contents of the documents cited and incorporated by reference herein), readily modify and/or adapt for various applications such specific embodiments, without undue experimentation, without departing from the general concept of the present invention. Such adaptations and modifications are therefore intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one skilled in the relevant art(s).

While various embodiments of the present invention have been described above, it should be understood that they have been presented by way of example, and not limitation. It would be apparent to one skilled in the relevant art(s) that various changes in form and detail could be made therein without departing from the spirit and scope of the invention. Thus, the present invention should not be limited by any of the above-described exemplary embodiments, but rather should be defined only in accordance with the following claims and their equivalents.

We claim:

1. A disposable drain bulb for the collection of fluid and other exudate, the disposable drain bulb comprising:
a drain interface with a one way valve for connection to a drain line through which the drain interface receives fluid and other exudate, the drain interface sealed with a capsule; and
a drain body, the drain body forming an internal reservoir into which the drain interface passes and that contains an absorbable material under vacuum while the drain interface remains in a closed state.

2. The disposable drain bulb of claim 1 wherein the capsule has one or more micro-perforations such that the one or more micro-perforations to rupture in the presence of sufficient pressure.

3. The disposable drain bulb of claim 2 wherein the drain interface enters an open state in response to the presence of sufficient pressure at the capsule causing the one or more micro-perforations to rupture.

4. The disposable drain bulb of claim 2 wherein the capsule contains a fluid.

5. The disposable drain bulb of claim 4 wherein the fluid is pulled in the drain body when the one or more micro-perforations rupture.

6. The disposable drain bulb of claim 1 wherein the absorbable material comprises a sponge.

7. A disposable drain bulb for the collection of fluid and other exudate, the disposable drain bulb comprising:
a drain interface with a one way valve for connection to a drain line through which the drain interface receives fluid and other exudate, the drain interface sealed with an actuatable valve; and
a drain body, the drain body forming an internal reservoir into which the drain interface passes and that contains an absorbable material in a vacuum while the drain interface remains in a closed state.

8. The disposable drain bulb of claim 7 wherein the actuatable valve moves to an open state in the presence of sufficient torque to open the valve.

9. The disposable drain bulb of claim 7 wherein the absorbable material comprises a sponge.
